(19) United States
(12) Patent Application Publication
(10) Pub. No.: US 2026/0345446 A1
(43) Pub. Date: Nov. 5, 2026

(54) WATER-IN-OIL TYPE EMULSIFYING AGENT AND WATER-IN-OIL TYPE EMULSIFIED COMPOSITION

(71) Applicant: NIKKO CHEMICALS CO., LTD., Chuo-ku, Tokyo (JP)

(72) Inventor: Rina ISHII, Chuo-ku, Tokyo (JP)

(21) Appl. No.: 19/485,333

(22) PCT Filed: May 29, 2024
(86) PCT No.: PCT/JP2024/019662

§ 371 Date: Nov. 18, 2025

(30) Foreign Application Priority Data
May 30, 2023 (JP) 2023-088726
May 30, 2023 (JP) 2023-088727

(51) Int. Cl. A61K 8/06; A61K 8/36; A61K 8/37; A61Q 19/00

(52) U.S. Cl. CPC A61K 8/064; A61K 8/361; A61K 8/375; A61Q 19/00; A61K 2800/10

(57) ABSTRACT

A water-in-oil type emulsifying agent having a metal soap and a (poly)glycerin fatty acid ester is provided. The water-in-oil type emulsifying agent exhibits good stability and usability without using organically modified clay minerals. The water-in-oil type emulsifying agent may also include an oil phase component and an aqueous phase component.

### FIELD OF THE INVENTION

The present invention relates to a water-in-oil type emulsifying agent and a water-in-oil type emulsified composition, especially a water-in-oil type emulsifying agent not using organically modified clay minerals and a water-in-oil type emulsified composition utilizing the same.

### BACKGROUND OF THE INVENTION

In the field of cosmetic preparations, water-in-oil type emulsified cosmetic preparations are used for creams, milky lotions, makeup cosmetic preparations, sunscreen cosmetic preparations, and others having a high moisturizing effect because they cover the surface of skin with an oil film, have the property of preventing moisture evaporation therefrom, or impart water resistance thereto. However, the problem with water-in-oil type emulsified cosmetic preparations is poor stability due to their high content of oily components constituting the external phase. Examples of conventional technologies to solve such a problem include the use of organically modified clay minerals having the effect of gelling oily components.

For example, JP-A 2014-172853 discloses a water-in-oil type emulsion cosmetic composition comprising an organically modified clay mineral, a hydrophobic silica, a silicone-type surfactant, an oil phase component, and water, and JP-A 2013-107865 discloses a water-in-oil type emulsified cosmetic preparation comprising an organically modified clay mineral, an ester-based nonionic surfactant with an HLB of 5 to 8 in which ethylene oxide is added, a nonionic surfactant with an HLB of less than 5 which is solid at room temperature, a nonionic surfactant with an HLB of less than 5 which is liquid at room temperature, waxes, a moisturizing agent, an oil phase component, and water. However, most organically modified clay minerals are obtained by reacting cationic surfactants that form organic cations such as organic ammonium ions or the like to make ion-exchange occur between inorganic cations contained in clay minerals and the organic cations. Therefore, the use of large amounts of organically modified clay minerals raises concerns from the viewpoint of environmental pollution or skin irritation caused by cationic surfactants. Further, these conventional technologies use silicone-based surfactants or ethylene oxide-added type nonionic surfactants. Accordingly, the surfactants in the conventional technologies also raise concerns from the viewpoint of biodegradability or skin irritation.

### SUMMARY OF THE INVENTION

The present invention was devised to address the above problems in the conventional technologies, and its purpose is to provide a water-in-oil type emulsifying agent not using organically modified clay minerals and a water-in-oil type emulsified composition with good stability and usability owing to the utilization of the same.

As a result of diligent research, the present inventors found that a water-in-oil type emulsifying agent using (A) a metal soap and (B) a (poly)glycerin fatty acid ester and a water-in-oil type emulsified composition utilizing the same were good in stability and usability, which led to the completion of the present invention.

The present invention can provide a water-in-oil type emulsified composition highly stable over time regardless of the polarity of oil phase components and excellent in usability such as good spreading or the like by using (A) a metal soap and (B) a (poly)glycerin fatty acid ester.

### EMBODIMENTS OF THE INVENTION

### Water-In-Oil Type Emulsifying Agent

In an aspect, the present invention provides a water-in-oil type emulsifying agent containing (A) a metal soap and (B) a (poly)glycerin fatty acid ester. (Poly)glycerin fatty acid ester (B) may be a (poly)glycerin fatty acid ester in which an average degree of polymerization of glycerin is 1 to 10 and the fatty acid is a linear unsaturated fatty acid and/or a branched fatty acid.

In an aspect of the present invention, metal soap (A) is the general name for salts of long-chain fatty acids with metals other than sodium and potassium. The type of metal soap is not particularly limited. In an aspect of the present invention, a metal salt constituting the metal soap is preferably a divalent metal salt. Examples include, for example, magnesium, calcium, strontium, zinc, barium, and others, and among these, magnesium and calcium are preferable. In the present invention, one or two of these can be used. Further, in the present invention, examples of a long-chain fatty acid constituting the metal soap include, for example, stearic acid, oleic acid, palmitic acid, lauric acid, myristic acid, a dimyristate, cetylphosphoric acid, ricinoleic acid, coconut fatty acid, octylic acid, and others. Specific examples of the metal soap include, for example, calcium behenate, magnesium stearate, calcium stearate, zinc stearate, magnesium palmitate, zinc palmitate, zinc laurate, magnesium laurate, zinc myristate, magnesium myristate, calcium myristate, calcium cetyl phosphate, zinc cetyl phosphate, zinc ricinoleate, magnesium cocoate, and others, and among these, magnesium stearate, calcium stearate, magnesium myristate, magnesium laurate, and zinc stearate, and others are preferable. In the present invention, one or two of these can be used.

In an aspect of the present invention, among metal soap (A), magnesium stearate refers to magnesium distearate ([CH₃(CH₂)₁₆COO]₂Mg), calcium stearate refers to calcium distearate ([CH₃(CH₂)₁₆COO]₂Ca), and magnesium myristate refers to magnesium dimyristate ([CH₃(CH₂)₁₂COO]₂Mg).

In an aspect of the present invention, a formulation amount of metal soap (A) contained in the water-in-oil type emulsifying agent is not particularly limited, but preferably 20.0 to 50.0 mass % of a total mass of the water-in-oil type emulsifying agent, and can be more preferably 20.0 to 35.0 mass % thereof.

In an aspect of the present invention, (poly)glycerin fatty acid ester (B) is a compound in which one or more hydroxyl groups in glycerin or polyglycerin are esterified with a fatty acid, and is preferably lipophilic. The fatty acid constituting (poly)glycerin fatty acid ester (B) is, for example, preferably a linear unsaturated fatty acid or a branched fatty acid with 18 carbons, and one or two or more selected from these can be used. In an example, examples of the unsaturated fatty acid or branched fatty acid with 18 carbons constituting (poly)glycerin fatty acid ester (B) include, isostearic acid, oleic acid, linoleic acid, ricinoleic acid, and others. Further, it may be a polymer like polyricinoleic acid.

In an aspect of the present invention, the (poly)glycerin constituting (poly)glycerin fatty acid ester (B) is glycerin or a polymer thereof, i.e., polyglycerin. The average degree of polymerization of the polyglycerin is preferably 1 to 10 and can be more preferably 1 to 6 in view of good stability.

In an aspect of the present invention, (poly)glycerin fatty acid ester (B) can be any (poly)glycerin fatty acid ester in which 1.0 mol of (poly)glycerin and 1.0 to 5.0 mol of a fatty acid form an ester bond. Among these, a (poly)glycerin fatty acid ester in which 1.0 mol of (poly)glycerin and 1.0 to 3.0 mol of a fatty acid form an ester bond is preferable in view of good stability.

An esterification rate of the (poly)glycerin fatty acid ester used in the present invention is 15 to 90% and preferably 25 to 85% in view of good stability.

In a method for calculating the esterification rate of the (poly)glycerin fatty acid ester used in the present invention, the rate is determined on the basis of the formula 2 from the average degree of polymerization (n) of polyglycerin calculated by the following formula 1 from the hydroxyl value according to the Japanese Standards of Quasi-Drug Ingredients and the number of moles (M) of fatty acid added to polyglycerin:

OHV=56110(n+2)/(74n+18) (formula 1)

Esterification rate (%)=(M/(n+2))×100 (formula 2)
OHV: hydroxyl value of polyglycerin
n: average degree of polymerization of polyglycerin
M: number of moles of fatty acid added to polyglycerin

In an aspect of the present invention, examples of (poly)glycerin fatty acid ester (B) are not particularly limited to, but include, for example, glyceryl isostearate, glyceryl oleate, glyceryl linoleate, glyceryl ricinoleate, polyglyceryl-2 isostearate, polyglyceryl-2 oleate, polyglyceryl-3 isostearate, polyglyceryl-3 oleate, polyglyceryl-3 polyricinoleate, polyglyceryl-4 isostearate, polyglyceryl-4 oleate, polyglyceryl-4 polyricinoleate, polyglyceryl-5 polyricinoleate, polyglyceryl-6 polyricinoleate, polyglyceryl-10 polyricinoleate, polyglyceryl-10 trioleate, and others. Among these, glyceryl isostearate, polyglyceryl-2 isostearate, polyglyceryl-4 oleate, polyglyceryl-6 polyricinoleate, polyglyceryl-10 polyricinoleate, polyglyceryl-10 trioleate, and others are preferable, and glyceryl isostearate, polyglyceryl-2 isostearate, and polyglyceryl-6 polyricinoleate are more preferable. In the present invention, one or two or more of these can be used.

In an aspect of the present invention, (poly)glycerin fatty acid ester (B) is not particularly limited by the intended application, but can be produced by a publicly-known method. Examples of commercially available products of polyglycerin fatty acid esters include, for example, NIKKOL MGIS (glyceryl isostearate), NIKKOL MGO (glyceryl myristate), NIKKOL DGMIS (polyglyceryl-2 isostearate), NIKKOL DGMO-90V (polyglyceryl-2 oleate), NIKKOL Tetraglyn 1-OV (polyglyceryl-4 oleate), NIKKOL Hexaglyn PR-15 (polygryceryl-6 polyricinoleate), and NIKKOL Decaglyn PR-20 (polyglyceryl-10 polyricinoleate) manufactured by Nikko Chemicals Co., Ltd., and others.

In an aspect, a formulation amount of (poly)glycerin fatty acid ester (B) contained in the water-in-oil type emulsifying agent of the present invention is not particularly limited, but preferably 50.0 to 80.0 mass % of a total mass of the water-in-oil type emulsifying agent, and can be more preferably 65.0 to 80.0 mass %.

In an aspect of the present invention, the agent can be formulated with metal soap (A) and (poly)glycerin fatty acid ester (B) at a mass ratio within a range of 1:1 to 1:10 in view of attaining good stability and usability, and can be formulated at preferably 1:1 to 1:4 and further preferably 1:2 to 1:4.

### Water-In-Oil Type Emulsified Composition

Further, in another aspect, the present invention provides a water-in-oil type emulsified composition using the water-in-oil type emulsifying agent described above. For example, this water-in-oil type emulsified composition may contain (A) a metal soap, (B) a (poly)glycerin fatty acid ester, (C) an oil phase component, and (D) an aqueous phase component, and the present invention can provide a water-in-oil type emulsified composition highly stable over time regardless of the polarity of oil phase components and excellent in usability such as spreading or the like.

In an aspect, metal soap (A) in the water-in-oil type emulsified composition may be the same as that which is described above in the water-in-oil type emulsifying agent. A formulation amount of metal soap (A) contained in the water-in-oil type emulsified composition is not particularly limited, but preferably 0.3 to 3 mass % of a total mass of the water-in-oil type emulsified composition, and can be more preferably 0.3 to 1 mass % thereof.

In an aspect, (poly)glycerin fatty acid ester (B) in the water-in-oil type emulsified composition may be the same as that which is described above in the water-in-oil type emulsifying agent. A formulation amount of (poly)glycerin fatty acid ester (B) contained in the water-in-oil type emulsified composition is not particularly limited, but preferably 0.7 to 5 mass % and more preferably 0.7 to 3 mass % of a total mass of the water-in-oil type emulsified composition.

In an aspect of the present invention, the water-in-oil type emulsified composition can be formulated with metal soap (A) and (poly)glycerin fatty acid ester (B) at a mass ratio within a range of 1:1 to 1:10 in view of attaining good stability and usability, and can be formulated at preferably 1:1 to 1:4 and further preferably 1:2 to 1:4.

In an aspect of the present invention, oil phase component (C) contained in the water-in-oil type emulsified composition is not particularly limited, but any oil phase component commonly used in cosmetic products can be suitably used. Specific examples include one or two or more of a hydrocarbon oil such as squalane, liquid paraffin, or the like, an ester oil such as cetyl ethylhexanoate, cetyl isooctanoate, triethylhexanoin, glyceryl tri (caprylate/caprate), or the like, a vegetable oil such as jojoba oil, olive oil, sunflower seed oil, apricot kernel oil, macadamia seed oil, coconut oil, or the like, a silicone oil such as diphenyl dimethicone, diphenylsiloxy phenyl trimethicone, or the like, and others.

In an example, a formulation amount of oil phase component (C) contained in the emulsified composition can be 10.0 to 60.0 mass % and preferably 10.0 to 40.0 mass % of a total mass of the water-in-oil type emulsified composition. Further, the composition can be formulated as necessary with a UV scattering agent such as fine titanium oxide particles, fine zinc oxide particles, or the like, or a coloring material such as an inorganic pigment, an organic pigment, or the like as a component other than oil contained in the oil phase component.

In an aspect of the present invention, aqueous phase component (D) contained in the water-in-oil type emulsified composition constitutes all the rest, and contains water as the main component. When the emulsified composition is applied to skin as a topical medication, a cosmetic preparation, or the like, water contained therein preferably contains no impurities, and thus, ion-exchanged water or pure water is preferably used. A component contained in aqueous phase component (D) other than water is not particularly limited, but any component commonly used in cosmetic products can be suitably used. Specific examples include, for example, a moisturizing agent, an emulsifying stabilizer, an antiseptic, a viscosity adjuster, a fragrance, and others.

In an aspect, examples of the moisturizing agent include glycerin, 1,3-butylene glycol, propylene glycol, dipropylene glycol, pentylene glycol, 1,3-propanediol, 1,6-hexanediol, 1,8-octanediol, caprylyl glycol, polyethylene glycol, and other polyhydric alcohols, sorbitol, xylitol, maltitol, and other sugars, amino acids, sodium hyaluronate, and others. Examples of the emulsifying stabilizer include magnesium sulfate, sodium chloride, calcium chloride, and others. Examples of the antiseptic include phenoxyethanol, phenylpropanol, 1,2-heptanediol, sodium benzoate, potassium sorbate, and others. Examples of the viscosity adjuster include xanthan gum, hydroxyethyl cellulose, hydroxypropyl methylcellulose stearoxy ether, guar gum, carbomer, (acrylates/alkyl acrylate (C10-30)) crosspolymer, and others.

In an aspect of the present invention, examples of dosage forms of the water-in-oil type emulsified composition are not particularly limited to, but include, for example, milky lotion form, paste form, gel form, cream form, and others.

In an aspect of the present invention, examples of a method for preparing the water-in-oil type emulsified composition are not particularly limited, but include, for example, a method including (1) a step of uniformly dispersing the oil phase component (component (C)) using the water-in-oil type emulsifying agent containing metal soap (A) and (poly)glycerin fatty acid ester (B) to obtain an oily composition or a step of emulsifying and dispersing components (A) to (C) at a time to obtain an oily composition, (2) a step of uniformly dissolving the aqueous phase component (component (D)) to obtain an aqueous composition, (3) a step of adding the aqueous composition to the oily composition, and (4) a step of stirring the oil phase and the aqueous phase so that they are emulsified to obtain the water-in-oil type emulsified composition.

In an aspect of the present invention, in the method for preparing the water-in-oil type emulsified composition described above, in step (1), the oil phase component is preferably dispersed at room temperature. In step (2), the aqueous phase component is preferably dissolved at room temperature. Step (3) is preferably performed at room temperature. In step (4), stirring is preferably performed at room temperature, and a common stirring device such as a homo disperser, a homo mixer, or the like may be used for stirring.

In an aspect of the present invention, the water-in-oil type emulsified composition obtained in the above manner is suitably used, for example, as a topical medication, a cosmetic preparation, or the like. Specific examples include, for example, an emollient milky lotion, a moisturizing cream, an emollient cream, a massage cream, a hand cream, a sunscreen milky lotion, a sunscreen cream, a liquid foundation, a cream foundation, and others.

The present invention is illustrated in more detail by giving examples below, but the present invention is not limited thereto. The formulation amounts in each example below are expressed as mass %.

### EXAMPLES

### Example 1. Method for Preparing Water-In-Oil Type Emulsifying Agent

### Preparation Method

The water-in-oil type emulsifying agents shown in Tables 1 to 2 were prepared. In a preparation method, metal soup (A) and (poly)glycerin fatty acid ester (B) were uniformly dispersed in a hot-water bath at 70 to 80 °C and then cooled to room temperature, thereby obtaining water-in-oil type emulsifying agents 1-1 to 1-13 as inventive products and comparative products 1-1 to 1-2.

TABLE 1

| | | Esterification | Water-in-oil type emulsifying agent | | | | | | | |
|---|---|---|---|---|---|---|---|---|---|---|
| Formulation component | | rate (%) | 1-1 | 1-2 | 1-3 | 1-4 | 1-5 | 1-6 | 1-7 | 1-8 |
| A | Aluminum distearate | | 17.00 | | | | | | | |
| | Magnesium stearate | | 17.00 | 17.00 | 17.00 | 17.00 | 17.00 | 20.00 | 34.00 | 50.00 |
| | Calcium stearate | | | 17.00 | | | | | | |
| | Magnesium myristate | | | | 17.00 | | | | | |
| | Magnesium laurate | | | | | 17.00 | | | | |
| | Zinc stearate | | | | | | 17.00 | | | |
| B | Glyceryl isostearate | 33.3 | 33.00 | 33.00 | 33.00 | 33.00 | 33.00 | 40.00 | 33.00 | 25.00 |
| | Polyglyceryl-6 polyricinoleate | 75.0 | 33.00 | 33.00 | 33.00 | 33.00 | 33.00 | 40.00 | 33.00 | 25.00 |
| | Polyglyceryl-2 isostearate | 25.0 | | | | | | | | |
| | Polyglyceryl-4 oleate | 16.7 | | | | | | | | |
| | Polyglyceryl-10 polyricinoleate | 83.3 | | | | | | | | |
| | Polyglyceryl-10 trioleate | 25.0 | | | | | | | | |
| | Polyglyceryl-2 distearate | 50.0 | | | | | | | | |
| | Polyglyceryl-10 pentastearate | 41.7 | | | | | | | | |

| | | | | | | | | |
|---|---|---|---|---|---|---|---|---|
| Total | 100.00 | 100.00 | 100.00 | 100.00 | 100.00 | 100.00 | 100.00 | 100.00 |

TABLE 2

| | | | | | | | Comparative | |
|---|---|---|---|---|---|---|---|---|
| | | Water-in-oil type emulsifying agent | | | | | product | |
| Formulation component | | 1-9 | 1-10 | 1-11 | 1-12 | 1-13 | 1-1 | 1-2 |
| A | Aluminum distearate | 17.00 | 17.00 | 17.00 | 17.00 | 13.00 | | 17.00 |
| | Magnesium stearate | 17.00 | 17.00 | 17.00 | 17.00 | 13.00 | | 17.00 |
| | Calcium stearate | | | | | | | |
| | Magnesium myristate | | | | | | | |
| | Magnesium laurate | | | | | | | |
| | Zinc stearate | | | | | | | |
| B | Glyceryl isostearate | | | | | 37.00 | 50.00 | |
| | Polyglyceryl-6 polyricinoleate | | | | | 37.00 | 50.00 | |
| | Polyglyceryl-2 isostearate | 33.00 | 33.00 | | | | | |
| | Polyglyceryl-4 oleate | | | 33.00 | 33.00 | | | |
| | Polyglyceryl-10 polyricinoleate | 33.00 | | 33.00 | | | | |
| | Polyglyceryl-10 trioleate | | 33.00 | | 33.00 | | | |
| | Polyglyceryl-2 distearate | | | | | | | 33.00 |
| | Polyglyceryl-10 pentastearate | | | | | | | 33.00 |

| | | | | | | | |
|---|---|---|---|---|---|---|---|
| Total | 100.00 | 100.00 | 100.00 | 100.00 | 100.00 | 100.00 | 100.00 |

### <Example 2. Evaluation of Water-In-Oil Type Emulsified Compositions Using Water-In-Oil Type Emulsifying Agents 1-1 to 1-13 and Those Using Comparative Products 1-1 to 1-2

### 1. Preparation Method

The water-in-oil type emulsified compositions shown in Tables 3 to 6 were prepared. In a preparation method, components constituting the first phase and those constituting the second phase were each weighed. The components constituting the first phase were each uniformly dispersed at room temperature. Note that in comparative example 2-4, each component constituting the first phase was uniformly dispersed in a hot-water bath at 70 to 80 °C and then cooled to room temperature. Next, the components constituting the second phase were each uniformly dissolved at room temperature. Further, the second phase was added to the first phase at room temperature, and they were stirred with a homo disperser so that they were emulsified, thus obtaining water-in-oil type emulsified compositions 2-1 to 2-20 and comparative examples 2-1 to 2-4.

### 2. Stability Evaluation

The day after preparation and after three months of storage at 45 °C, the appearance of each composition was visually observed in line with the following evaluation criteria. Note that this evaluation was performed for evaluating the stability of each water-in-oil type emulsified composition, thereby simultaneously checking the function of the water-in-oil type emulsifying agent.

### <<Evaluation Criteria: Stability>>

A: separation such as oil floating, creaming, or the like did not occur
B: separation such as oil floating, creaming, or the like was observed

TABLE 3

| | | Those formulated with metal soap | | | | | |
|---|---|---|---|---|---|---|---|
| | | Water-in-oil type emulsified | | | Those formulated with no metal soap | | |
| | | composition | | | Comparative example | | |
| Formulation component | | 2-1 | 2-2 | 2-3 | 2-1 | 2-2 | 2-3 |
| First | Water-in-oil type | 3.00 | 3.00 | 3.00 | | | |
| phase | emulsifying agent 1-3 | | | | | | |
| | Comparative product 1-1 | | | | 2.00 | 2.00 | 2.00 |
| | Squalane | 25.00 | | | 25.00 | | |
| | Glyceryl | | 25.00 | | | 25.00 | |
| | tri(caprylate/caprate) | | | | | | |
| | Cetylethylhexanoate | | | 25.00 | | | 25.00 |
| Second | Magnesium sulfate | 0.40 | 0.40 | 0.40 | 0.40 | 0.40 | 0.40 |
| phase | Glycerin | 5.00 | 5.00 | 5.00 | 5.00 | 5.00 | 5.00 |
| | 1,3-propane diol | 5.00 | 5.00 | 5.00 | 5.00 | 5.00 | 5.00 |
| | Antiseptic | Appropriate | Appropriate | Appropriate | Appropriate | Appropriate | Appropriate |
| | | amount | amount | amount | amount | amount | amount |
| | Water | All the rest | All the rest | All the rest | All the rest | All the rest | All the rest |

| | | | | | | |
|---|---|---|---|---|---|---|
| Total | 100.00 | 100.00 | 100.00 | 100.00 | 100.00 | 100.00 |
| Observation of appearance | A | A | A | A | A | A |
| the day after preparation | | | | | | |
| Observation of appearance | A | A | A | B | B | B |
| after three months at 45 °C | | | | | | |

As shown by the results in Table 3, compositions 2-1 to 2-3 which used a water-in-oil type emulsifying agent formulated with metal soap (A) all showed no separation and appeared uniform the day after preparation. Further, they were also stable after three months of storage at 45 °C as well. On the other hand, comparative examples 2-1 to 2-3 which used a water-in-oil type emulsifying agent formulated with no metal soap all showed no separation the day after preparation, but were separated after three months of storage at 45 °C. This fact demonstrates that emulsified compositions highly stable over time are obtained when the compositions are formulated with metal soap (A) and (poly)glycerin fatty acid ester (B).

TABLE 4

| | | Those with varying types or amounts of metal soap | | | | | | |
|---|---|---|---|---|---|---|---|---|
| | | Water-in-oil type emulsified composition | | | | | | |
| Formulation component | | 2-4 | 2-5 | 2-6 | 2-7 | 2-8 | 2-9 | 2-10 |
| First | Water-in-oil type | 3.00 | | | | | | |
| phase | emulsifying agent 1-2 | | | | | | | |
| | Water-in-oil type | | 3.00 | | | | | |
| | emulsifying agent 1-3 | | | | | | | |
| | Water-in-oil type | | | 3.00 | | | | |
| | emulsifying agent 1-4 | | | | | | | |
| | Water-in-oil type | | | | 3.00 | | | |
| | emulsifying agent 1-5 | | | | | | | |
| | Water-in-oil type | | | | | 2.50 | | |
| | emulsifying agent 1-6 | | | | | | | |
| | Water-in-oil type | | | | | | 3.00 | |
| | emulsifying agent 1-7 | | | | | | | |
| | Water-in-oil type | | | | | | | 5.00 |
| | emulsifying agent 1-8 | | | | | | | |
| | Glyceryl | 25.00 | 25.00 | 25.00 | 25.00 | 25.00 | 25.00 | 25.00 |
| | tri(caprylate/caprate) | | | | | | | |
| Second | Magnesium sulfate | 0.40 | 0.40 | 0.40 | 0.40 | 0.40 | 0.40 | 0.40 |
| phase | Glycerin | 5.00 | 5.00 | 5.00 | 5.00 | 5.00 | 5.00 | 5.00 |
| | 1,3-propane diol | 5.00 | 5.00 | 5.00 | 5.00 | 5.00 | 5.00 | 5.00 |
| | Antiseptic | Appropriate | Appropriate | Appropriate | Appropriate | Appropriate | Appropriate | Appropriate |
| | | amount | amount | amount | amount | amount | amount | amount |
| | Water | All the rest | All the rest | All the rest | All the rest | All the rest | All the rest | All the rest |

| | | | | | | | |
|---|---|---|---|---|---|---|---|
| Total | 100.00 | 100.00 | 100.00 | 100.00 | 100.00 | 100.00 | 100.00 |
| Observation of appearance | A | A | A | A | A | A | A |
| the day after preparation | | | | | | | |
| Observation of appearance | A | A | A | A | A | A | A |
| after three months at 45 °C | | | | | | | |

As shown by the results in Table 4, compositions 2-2 and 2-4 to 2-7 which used water-in-oil type emulsifying agents with varying types of metal soap (A) all showed no separation and were stable the day after preparation and after three months of storage at 45 °C. Further, water-in-oil type emulsified compositions 2-8 to 2-10 with varying amounts of metal soap (A) from 0.5 to 3 mass % also showed no separation and were stable the day after preparation and after three months of storage at 45 °C as well. This fact demonstrates that emulsified compositions highly stable over time are obtained when appropriate types or amounts of metal soap (A) are used in the product of the present invention.

TABLE 5

| | | Those with varying types or amounts of (poly)glycerin fatty acid ester | | | | | |
|---|---|---|---|---|---|---|---|
| | | | | | | | Comparative |
| | | Water-in-oil type emulsified composition | | | | | example |
| Formulation component | | 2-11 | 2-12 | 2-13 | 2-14 | 2-15 | 2-4 |
| First | Water-in-oil type | 3.00 | | | | | |
| phase | emulsifying agent 1-9 | | | | | | |
| | Water-in-oil type | | 3.00 | | | | |
| | emulsifying agent 1-10 | | | | | | |
| | Water-in-oil type | | | 3.00 | | | |
| | emulsifying agent 1-11 | | | | | | |
| | Water-in-oil type | | | | 3.00 | | |
| | emulsifying agent 1-12 | | | | | | |
| | Water-in-oil type | | | | | 4.00 | |
| | emulsifying agent 1-13 | | | | | | |
| | Comparative product 1-2 | | | | | | 3.00 |
| | Glyceryl | 25.00 | 25.00 | 25.00 | 25.00 | 25.00 | 25.00 |
| | tri(caprylate/caprate) | | | | | | |
| Second | Magnesium sulfate | 0.40 | 0.40 | 0.40 | 0.40 | 0.40 | 0.40 |
| phase | Glycerin | 5.00 | 5.00 | 5.00 | 5.00 | 5.00 | 5.00 |
| | 1,3-propane diol | 5.00 | 5.00 | 5.00 | 5.00 | 5.00 | 5.00 |
| | Antiseptic | Appropriate | Appropriate | Appropriate | Appropriate | Appropriate | Appropriate |
| | | amount | amount | amount | amount | amount | amount |
| | Water | All the rest | All the rest | All the rest | All the rest | All the rest | All the rest |

| | | | | | | |
|---|---|---|---|---|---|---|
| Total | 100.00 | 100.00 | 100.00 | 100.00 | 100.00 | 100.00 |
| Observation of appearance | A | A | A | A | A | B |
| the day after preparation | | | | | | |
| Observation of appearance | A | A | A | A | A | B |
| after three months at 45 °C | | | | | | |

As shown by the results in Table 5, water-in-oil type emulsified compositions 2-11 to 2-14 with varying types of (poly)glycerin fatty acid ester (B) all showed no separation and were stable the day after preparation and after three months of storage at 45 °C. Further, composition 2-15 which used a water-in-oil type emulsifying agent with an increased amount of (poly)glycerin fatty acid ester (B) also showed no separation and was stable the day after preparation and after three months of storage at 45 °C as well. On the other hand, comparative example 2-4 which used (poly)glycerin fatty acid ester (B) having a linear saturated fatty acid was separated the day after preparation and after three months of storage at 45 °C. This fact demonstrates that emulsified compositions highly stable over time are obtained when a specific (poly)glycerin fatty acid ester (B) is used in the product of the present invention.

TABLE 6

| | | Those with varying amounts of oil | | | | |
|---|---|---|---|---|---|---|
| | | Water-in-oil type emulsified composition | | | | |
| Formulation component | | 2-16 | 2-17 | 2-18 | 2-19 | 2-20 |
| First | Water-in-oil type | 3.00 | 3.00 | 3.00 | 3.00 | 3.00 |
| phase | emulsifying agent 1-3 | | | | | |
| | Squalane | 10.00 | | | 50.00 | |
| | Glyceryl tri(caprylate/caprate) | | 10.00 | | | 50.00 |
| | Cetylethylhexanoate | | | 10.00 | | |
| Second | Magnesium sulfate | 0.40 | 0.40 | 0.40 | 0.40 | 0.40 |
| phase | Glycerin | 5.00 | 5.00 | 5.00 | 5.00 | 5.00 |
| | 1,3-propanediol | 5.00 | 5.00 | 5.00 | 5.00 | 5.00 |
| | Antiseptic | Appropriate | Appropriate | Appropriate | Appropriate | Appropriate |
| | | amount | amount | amount | amount | amount |
| | Water | All the rest | All the rest | All the rest | All the rest | All the rest |

| | | | | | |
|---|---|---|---|---|---|
| Total | 100.00 | 100.00 | 100.00 | 100.00 | 100.00 |
| Observation of appearance | A | A | A | A | A |
| the day after preparation | | | | | |
| Observation of appearance | A | A | A | A | A |
| after three months at 45 °C | | | | | |

As shown by the results in Table 6, water-in-oil type emulsified compositions 2-16 to 2-20 with varying amounts of oil from 10 to 50 mass % all showed no separation and were stable the day after preparation and after three months of storage at 45 °C. This fact demonstrates that emulsified compositions highly stable over time are obtained for a wide range of oil amounts in the product of the present invention.

Examples of water-in-oil type emulsified compositions formulated with the water-in-oil type emulsifying agent of the product of the present invention are illustrated below. It was able to be confirmed by the evaluation method in example 2 that the compositions of examples 3 to 7 were all excellent in stability. The formulation amounts are expressed as mass %.

Example 3: Milky lotion

| First phase: | Water-in-oil type emulsifying | 4.00 |
|---|---|---|
| | agent 1-1 | |
| | Squalane | 6.00 |
| | Methylheptylisostearate | 15.00 |
| | Retinol | 0.20 |
| | Fragrance | Appropriate amount |
| Second phase: | 1,3-butylene glycol | 5.00 |
| | Sodium chloride | 0.10 |
| Sodium acrylates cross polymer-2 | | 3.00 |
| | LPA* | 0.20 |
| | Antiseptic | Appropriate amount |
| | Purified water | All the rest |
| Total | | 100.00 |

*LPA: lecithin, lysolecithin, lysophosphatidic acid (manufactured by Nikko Chemicals Co., Ltd.)

LPA: lecithin, lysolecithin, lysophosphatidic acid (manufactured by Nikko Chemicals Co., Ltd.)

Preparation method: after the first phase was stirred with a dispersion mixer, the second phase was gradually added and stirred, and then preparation ended.

Result: the product was uniform both the day after preparation and after three months of storage at 45 °C.

Example 4: skin care cream

| | | |
|---|---|---|
| First phase: | Water-in-oil type emulsifying | 5.00 |
| | agent 1-2 | |
| | Triethylhexanoin | 7.00 |
| | Ethylhexyl palmitate | 0.50 |
| | Cetyl ethylhexanoate | 0.50 |
| | Squalane | 2.00 |
| | Jojoba seed oil | 2.00 |
| | Meadowfoam seed oil | 2.00 |
| | Cyclopentasiloxane | 2.00 |
| | NIKKOL WOOD WAX* | 5.00 |
| | NIKKOL NIKKOWAX LM* | 2.00 |
| | Microcrystalline wax | 1.00 |
| | Antiseptic | Appropriate amount |
| Second phase: | 1,3-butylene glycol | 2.00 |
| | Glycerin | 10.00 |
| | Antiseptic | Appropriate amount |
| Third phase: | Xanthan gum | 0.10 |
| | Dipotassium glycyrrhizinate | 0.10 |
| | EDTA-2Na | 0.05 |
| | Purified water | All the rest |
| Total | | 100.00 |

*NIKKOL WOOD WAX: dextrin palmitate, (myristic acid/palmitic acid/stearic acid/ricinoleic acid) glycerides (manufactured by Nikko Chemicals Co., Ltd.)
*NIKKOL NIKKOWAX LM: (hydroxystearic acid/stearic acid/eicosadienoic acid) polyglyceryl-10, dextrin palmitate (manufactured by Nikko Chemicals Co., Ltd.)

Preparation method: after the second phase was uniformly dissolved in a hot-water bath at 70 to 80 °C, the second phase was added and uniformly mixed to the third phase. Further, while the first phase was being stirred with a dispersion mixer, the second phase and the third phase were gradually added and stirred, and then preparation ended.

Result: the product was uniform both the day after preparation and after three months of storage at 45 °C.

Example 5: eye cream

| | | |
|---|---|---|
| First phase: | Glycerin | 10.00 |
| | 1,3-propanediol | 5.00 |
| | LPA | 0.10 |
| | Sodium hyaluronate | 0.01 |
| | Magnesium ascorbyl phosphate | 0.50 |
| | Antiseptic | Appropriate amount |
| | Purified water | All the rest |
| Second phase: | Magnesium stearate | 1.70 |
| | Glyceryl isostearate | 1.65 |
| | Polyglyceryl-6 polyricinoleate | 1.65 |
| | Glyceryl tri(caprylate/caprate) | 25.00 |
| | Polyglyceryl-10 decamacadamiate | 5.00 |
| | Tocopherol | 0.10 |
| | Fragrance | Appropriate amount |
| Total | | 100.00 |

Preparation method: the first phase was stirred at room temperature and made to be uniform. Further, while the second phase was being stirred with a dispersion mixer, the first phase was gradually added and stirred, and then preparation ended.

Result: the product was uniform both the day after preparation and after three months of storage at 45 °C.

Example 6: liquid foundation

| | | |
|---|---|---|
| First phase: | Aluminum distearate | 0.85 |
| | Magnesium stearate | 0.85 |
| | Polyglyceryl-2 isostearate | 1.65 |
| | Polyglyceryl-10 polyricinoleate | 1.65 |
| | Isododecane | 5.00 |
| | TNP50T7* | 10.00 |
| | X-21-5595* | 3.00 |
| | Diphenylsiloxy phenyl trimethicone | 4.50 |
| | Meadowfoam oil | 1.00 |
| | D-400* | 2.00 |
| | EM SERICITE EW-F8* | 1.00 |
| | FDP-C-WHITE3* | 7.20 |
| | FDP-C-RED3* | 1.20 |
| | FDP-C-YELLOW3* | 2.40 |
| | FDP-C-BLACK3* | 1.20 |
| | Antiseptic | Appropriate amount |
| | Fragrance | Appropriate amount |
| Second phase: | Ethylhexyl methoxysilicate | 5.00 |
| | Bis-ethylhexyloxyphenol methoxyphenyl triazine | 2.00 |
| | Diethylamino hydroxybenzoyl hexyl benzoate | 2.00 |
| Third phase: | Niacinamide | 3.00 |
| | Purified water | All the rest |
| Total | | 100.00 |

*TNP50T7: alkyl benzoate (C12-15), titanium oxide, alumina, methicone, polyhydroxystearic acid (manufactured by Kobo Dispatek, inc.)
*X-21-5595: trimethylsiloxysilicate, silica (manufactured by Shin-Etsu Chemical Co., Ltd.)
*D-400: (HDI/trimethylol hexyllactone) crosspolymer, silica (manufactured by TOSHIKI PIGMENT CO., LTD.)
*EM SERICITE EW-F8: mica, aloe vera leaf extract, coconut oil (manufactured by TOSHIKI PIGMENT CO., LTD.)
*FDP-C-WHITE3: titanium oxide, aluminum hydroxide, talc, hydrogen dimethicone (manufactured by TOSHIKI PIGMENT CO., LTD.)
*FDP-C-RED3: titanium oxide, aluminum hydroxide, talc, hydrogen dimethicone, iron oxide (manufactured by TOSHIKI PIGMENT CO., LTD.)
*FDP-C-YELLOW3: titanium oxide, aluminum hydroxide, talc, hydrogen dimethicone, iron oxide (manufactured by TOSHIKI PIGMENT CO., LTD.)
*FDP-C-BLACK3: titanium oxide, aluminum hydroxide, talc, hydrogen dimethicone, iron oxide (manufactured by TOSHIKI PIGMENT CO., LTD.)

Preparation method: the second phase was uniformly dissolved in a hot-water bath at 70 to 80 °C and then cooled to room temperature. After the second phase was added and uniformly mixed to the first phase, they were stirred with a homo mixer and made to be uniform. Next, the third phase was uniformly dissolved in a hot-water bath at 70 to 80 °C and then cooled to room temperature. Further, while the first phase and the second phase were being stirred with a homo mixer, the third phase was added and stirred, and then preparation ended.

Result: the product was uniform both the day after preparation and after three months of storage at 45 °C.

Example 7: sunscreen milk

| | | |
|---|---|---|
| First phase: | Aluminum distearate | 0.85 |
| | Magnesium stearate | 0.85 |
| | Polyglyceryl-4 oleate | 1.70 |
| | Polyglyceryl-10 trioleate | 1.70 |
| | Isopropyl isostearate | 20.00 |
| | Cyclopentasiloxane | 5.00 |
| | IOPP60ZIAJ* | 15.00 |
| | Silica | 0.50 |
| | D-400 | 1.50 |
| | KTZ SHIMMER WHITE-12* | 1.50 |
| | Talc | 2.00 |
| | Antiseptic | Appropriate amount |
| | Fragrance | Appropriate amount |
| Second phase: | 1,3-propanediol | 5.00 |
| | Glycerin | 2.00 |
| | Biosaccharide gum-4 | 0.30 |
| | Purified water | 6.70 |
| Third phase: | Zinc glycinate | 1.00 |
| | Aspartic acid | 0.20 |
| | Purified water | All the rest |
| Total | | 100.00 |

*IOPP60ZIAJ: zinc oxide, ethylhexyl palmitate, isostearic acid, polyhydroxystearic acid (manufactured by Kobo Dispatek, inc.)
*KTZ SHIMMER WHITE-12: mica, titanium oxide, isopropyl titanium triisostearate (manufactured by Kobo Dispatek, inc.)

Preparation method: after the first to third phases were each uniformly mixed at room temperature, the third phase was added and uniformly mixed to the second phase. Further, while the first phase was being stirred with a homo disperser, the second+third phase was added and stirred, and then preparation ended. Result: the product was uniform both the day after preparation and after three months of storage at 45 °C.

### INDUSTRIAL APPLICABILITY

The product of the present invention uses (A) a metal soap and (B) a (poly)glycerin fatty acid ester, whereby a water-in-oil type emulsifying agent not using organically modified clay minerals, and a water-in-oil type emulsified composition with good stability and usability utilizing the same can be provided.

1. A water-in-oil type emulsifying agent comprising the following components (A) and (B):
(A) a metal soap; and
(B) a (poly)glycerin fatty acid ester in which an average degree of polymerization of glycerin is 1 to 10 and a fatty acid thereof is a linear unsaturated fatty acid and/or a branched fatty acid.

2. The water-in-oil type emulsifying agent according to claim 1, wherein a metal salt of the metal soap of the component (A) comprises at least one of magnesium and calcium.

3. The water-in-oil type emulsifying agent according to claim 1, wherein the metal soap of the component (A) comprises one or more selected from the group consisting of calcium behenate, magnesium stearate, calcium stearate, zinc stearate, magnesium palmitate, zinc palmitate, zinc laurate, magnesium laurate, zinc myristate, magnesium myristate, calcium myristate, calcium cetyl phosphate, zinc cetyl phosphate, zinc ricinoleate, and magnesium cocoate.

4. The water-in-oil type emulsifying agent according to claim 1, wherein a formulation amount of the metal soap of the component (A) is 20.0 to 50.0 mass % of a total mass of the water-in-oil type emulsifying agent.

5. The water-in-oil type emulsifying agent according to claim 1, wherein the fatty acid of the (poly)glycerin fatty acid ester of the component (B) is one or more selected from the group consisting of isostearic acid, oleic acid, linoleic acid, ricinoleic acid, and polyricinoleic acid.

6. The water-in-oil type emulsifying agent according to claim 1, wherein the (poly)glycerin fatty acid ester of the component (B) is one or more selected from the group consisting of glyceryl isostearate, polyglyceryl-2 isostearate, polyglyceryl-4 oleate, polyglyceryl-6 polyricinoleate, polyglyceryl-10 polyricinoleate, and polyglyceryl-10 trioleate.

7. The water-in-oil type emulsifying agent according to claim 1, wherein a formulation amount of the (poly)glycerin fatty acid ester of the component (B) is 50.0 to 80.0 mass % of a total mass of the water-in-oil type emulsifying agent.

8. A water-in-oil type emulsified composition comprising the water-in-oil type emulsifying agent according to claim 1.

9. A water-in-oil type emulsified composition comprising the following components (A) to (D):
(A) a metal soap;
(B) a (poly)glycerin fatty acid ester in which an average degree of polymerization of glycerin is 1 to 10 and a fatty acid thereof is a linear unsaturated fatty acid and/or a branched fatty acid;
(C) an oil phase component; and
(D) an aqueous phase component.

10. The water-in-oil type emulsified composition according to claim 9, wherein a metal salt of the metal soap of the component (A) comprises at least one of magnesium and calcium.

11. The water-in-oil type emulsified composition according to claim 9, wherein the metal soap of the component (A) comprises one or more selected from the group consisting of calcium behenate, magnesium stearate, calcium stearate, zinc stearate, magnesium palmitate, zinc palmitate, zinc laurate, magnesium laurate, zinc myristate, magnesium myristate, calcium myristate, calcium cetyl phosphate, zinc cetyl phosphate, zinc ricinoleate, and magnesium cocoate.

12. The water-in-oil type emulsified composition according to claim 9, wherein a formulation amount of the metal soap of the component (A) is 0.3 to 3 mass % of a total mass of the water-in-oil type emulsified composition.

13. The water-in-oil type emulsified composition according to claim 9, wherein the fatty acid of the (poly)glycerin fatty acid ester of the component (B) is one or more selected from the group consisting of isostearic acid, oleic acid, linoleic acid, ricinoleic acid, and polyricinoleic acid.

14. The water-in-oil type emulsifying agent according to claim 9, wherein the (poly)glycerin fatty acid ester of the component (B) is one or more selected from the group consisting of glyceryl isostearate, polyglyceryl-2 isostearate, polyglyceryl-4 oleate, polyglyceryl-6 polyricinoleate, polyglyceryl-10 polyricinoleate, and polyglyceryl-10 trioleate.

15. The water-in-oil type emulsified composition according to claim 9, wherein a formulation amount of the (poly)glycerin fatty acid ester of the component (B) is 0.7 to 5 mass % of a total mass of the water-in-oil type emulsified composition.

16. The water-in-oil type emulsified composition according to claim 9, wherein the oil phase component (C) is one or two or more selected from the group consisting of a hydrocarbon oil, an ester oil, a vegetable oil, and a silicone oil.

17. The water-in-oil type emulsified composition according to claim 9, wherein a formulation amount of the oil phase component (C) is 10.0 to 60.0 mass % of a total mass of the water-in-oil type emulsified composition.
